(19) United States
(12) Patent Application Publication
(10) Pub. No.: US 2025/0072907 A1
(43) Pub. Date: Mar. 6, 2025

(54) Flexible Instrument and Methods of Using and Manufacturing the Flexible Instrument

(71) Applicant: Howmedica Osteonics Corp., Mahwah, NJ (US)

(72) Inventor: Heinrich George Crous, Cork (IE)

(21) Appl. No.: 18/824,509

(22) Filed: Sep. 4, 2024

Related U.S. Application Data
(60) Provisional application No. 63/536,580, filed on Sep. 5, 2023.

(51) Int. Cl. A61B 17/16

(52) U.S. Cl. CPC A61B 17/1631; A61B 2217/00

(57) ABSTRACT

A flexible instrument includes a first end portion, a second end portion opposite the first end portion, and a shaft extending from the first end portion to the second end portion along a central longitudinal axis. The shaft includes a first helical segment extending from the first end portion to the second end portion along a first helical path about the central longitudinal axis. The second helical segment extends from the first end portion to the second end portion along a second helical path about the central longitudinal axis. The first helical segment includes a first plurality of contact surfaces and the second helical segment includes a second plurality of contact surfaces such that when a torque is applied to the shaft, the first plurality of contact surfaces or the second plurality of contact surfaces bears against the other.

### CROSS-REFERENCE TO RELATED APPLICATIONS

This application claims the benefit of the filing date of U.S. Provisional Patent Application No. 63/536,580 filed on Sep. 5, 2023, the disclosure of which is hereby incorporated by reference in its entirety.

### BACKGROUND

Tools and instruments used for reaming bone during surgery are often navigated through or around tight spaces which often requires the tool to bend or flex to reach such spaces. Reamer tools may also include rotatable reamer shafts, e.g., driven by a handheld and/or motorized device, to then form a hole or bore in the bone, where the ability to transfer torque from the device to the reamer of the reamer tool is essential. As such, a reamer tool with a reamer that is rotatable (i.e., transfers torque) while the reamer is angulated may be desirable to reach a desired target surgical site or to otherwise operate proximate the desired target surgical site.

Existing reamer tools may be single or multi-component assemblies with generally solid reamer structures. For added flexibility, such reamers may be fabricated so that a limited amount of material is removed from the solid structure. Conventional subtractive manufacturing techniques may be used to form such a reamer. However, the aforementioned reamer tools may have limited capacity to bend and/or transfer torsional forces, particularly at the same time.

Accordingly, a reamer with a capacity to bend while also having improved load transfer performance is desirable for improving the efficacy of surgical procedures.

### BRIEF SUMMARY

The intended use of the medical tool disclosed herein is to provide torsional load through a circular cannulated shaft, the shaft being manipulable from a linear shape to being bent at an oblique angle where the angle at a free end of the flexible shaft may be different from the angle at a base of the flexible shaft, the angle being relative to a central longitudinal axis through the shaft when the shaft is in a neutral state with the linear shape. The shaft allows flexing ability while still allowing the transfer of torque through the shaft at oblique angles. The tool may be additively manufactured as a single monolithic structure. The shaft of the tool may be cannulated with specific profiled geometry between inner and outer boundary walls of the shaft. The shaft may have a geometry such that a series of helical spirals connect a proximal and a distal tip of the shaft. The helical spirals allow the shaft to flex when force is applied from a direction transverse to the longitudinal axis of the shaft. Along each helical spiral exists a series of vertical columns each spaced apart by a slot. The helical spirals may be arranged so that vertical columns of different spirals may form a vertical line of spaced apart vertical columns. Together these vertical columns allow torsional load to be transferred between the helical spirals and between the proximal tip and the distal tip independent of a given angulation of the shaft while such torsional loads are applied.

According to a first aspect of the disclosure, a flexible instrument includes a first end portion in a first rotational position, a second end portion opposite the first end portion, the second end portion in a second rotational position relative to the first rotational position when the flexible instrument is at rest, and a cannulated shaft extending along a central longitudinal axis from the first end portion to the second end portion, the cannulated shaft being deformable to vary a curvature of the central longitudinal axis. The flexible instrument may be configured to be rotated by the first end portion, and the second end portion may be configured to maintain the second rotational position during rotation of the flexible instrument. The first end portion, the second end portion and the cannulated shaft may be formed monolithically.

According to a second aspect of the disclosure, a flexible instrument includes a first end portion, a second end portion opposite the first end portion, and a shaft. The shaft may extend from the first end portion to the second portion along a central longitudinal axis. The shaft may include a first helical segment and a second helical segment. The first helical segment may extend from the first end portion to the second end portion along a first helical path about the central longitudinal axis. The second helical segment may extend from the first end portion to the second end portion along a second helical path about the central longitudinal axis. The first helical segment may include a first plurality of contact surfaces and the second helical segment may include a second plurality of contact surfaces such that when a torque is applied to the shaft, the first plurality of contact surfaces or the second plurality of contact surfaces bears against the other of the first plurality of contact surfaces or the second plurality of contact surfaces.

Further in the flexible instrument according to the second aspect of the disclosure, the first helical segment may include a protrusion extending from the first helical segment. The second helical segment may define a recess on a face of the second helical segment configured to receive the protrusion of the first helical segment. The first plurality of contact surfaces may further include a plurality of protrusions extending from the first helical segment, wherein the plurality of protrusions is spaced apart from each other along a length of the first helical segment. The plurality of protrusions may extend from the first helical segment in parallel direction. The second plurality of contact surfaces may further include a plurality of recesses on a face of the second helical segment, the plurality of recesses spaced apart from each other along a length of the second helical segment. Each one of the plurality of protrusions may be positioned within a corresponding one of the plurality of recesses. The first helical segment may define a plurality of recesses on a face of the first helical segment. The instrument may further include a plurality of protrusions extending from the second helical segment. Each of the plurality of protrusions extending from the second helical segment may be positioned within a corresponding one of the plurality of recesses defined by the first helical segment. The plurality of protrusions and the plurality of recesses may alternate along the length of the first helical segment. The protrusion may extend in a direction parallel to the longitudinal axis.

Further in the flexible instrument according to the second aspect of the disclosure, the shaft may further include a plurality of additional helical segments, the first, second and plurality of additional helical segments defining a cannulation of the shaft. The shaft may be perforated defining gaps between the first helical segment and the second helical segment. The first end portion and the second portion may be solid such that outer surfaces thereof have no gaps. The recess may be defined on a radially outer face of the second helical segment. The recess may be defined on a radially inner face of the second helical segment. The first end portion, the shaft, and the second end portion are monolithic. The recess may be sized and shaped to allow movement of the protrusion relative to the recess when the instrument is flexed. The flexible instrument may be formed of titanium of stainless steel. The flexible instrument may be formed of plastic. The first helical segment may define a pitch angle relative to a plane perpendicular to the central longitudinal axis of at least 35 degrees. The shaft may be cannulated. The flexible instrument may be a reaming tool. The instrument may further include a handle for coupling to a reaming instrument. The instrument may further include a cutting tip extending from the first end portion, wherein the cutting tip is configured to resect bone. The flexible instrument may be additively manufactured.

According to a third aspect of the disclosure, a flexible instrument may include a first end portion, a second end portion opposite the first end portion, and a cannulated shaft. The shaft may extend from the first end portion to the second end portion such that the cannulated shaft, the first end portion and the second end portion are aligned along a central longitudinal axis. The cannulated shaft may have neutral configuration where the central longitudinal axis is linear and the cannulated shaft may be deformable into a bent configuration such that the central longitudinal axis is curved. The cannulates shaft may be adapted to transfer torque between surfaces of interfacing subportions of the cannulated shaft when the cannulated shaft is rotated about the central longitudinal axis, the surfaces of the interfacing subportions being proximate an outer surface of the cannulated shaft and remote from the central longitudinal axis.

Further in the flexible instrument according to the third aspect of the disclosure, the surfaces of the interfacing subportions may be on respective planes that are parallel to the central longitudinal axis. The cannulated shaft may be formed of a plurality of segments extending from the first end portion to the second end portion. Each of the plurality of segments may revolve around the central longitudinal axis as each segment extends from the first end portion to the second portion. The plurality of segments may be parallel to one another. Adjacent ones of the plurality of segments may be interconnected. The interconnection may be is sliding contact between a first of the plurality of segments and a second of the plurality of segments. Each of the plurality of segments may include a protrusion along a length and defines a recess separated from the protrusion along the length. The protrusion of each of the plurality of segments may mate with the recess of an adjacent one of the plurality of segments. The flexible instrument may further include a handle and a cutting tip. The handle may be at the first end portion or the second end portion. The cutting tip may be at the other of the first end portion or the second end portion and may be configured to resect bone. The flexible instrument may be additively manufactured.

According to a fourth aspect of the disclosure, a flexible instrument may include a first end portion, a second end portion opposite the first end portion, and a shaft. The shaft may extend along a central longitudinal axis from the first end portion to the second end portion. The shaft may include a plurality of parallel segments extending from the first end portion to the second end portion. Each of the parallel segments may be shaped to revolve around the central longitudinal axis as the segments extend from the first end portion the second portion.

Further in the flexible instrument according to the fourth aspect of the disclosure, each of the segments may be operatively connected with an adjacent segment. The first end portion, the second end portion and the shaft may be formed monolithically. The plurality of parallel segments may define a cannulation through the shaft long the central longitudinal axis. The flexible instrument may further include a handle and a cutting tip. The handle may be at the first end portion or the second end portion. The cutting tip may be at the other of the first end portion or the second end portion. The cutting tip may be configured to resect bone.

According to a fifth aspect of the disclosure, a method of manufacturing a three-dimensional flexible instrument may include additively manufacturing the flexible instrument, wherein the flexible instrument includes a first end portion, a second end portion opposite the first end portion, and a shaft. The shaft may extend from the first end portion to the second end portion along a central longitudinal axis. The shaft may include a first helical segment and a second helical segment. The first helical segment may extend from the first end portion to the second portion along a first helical path about the central longitudinal axis. The second helical segment may extend from the first end portion to the second end portion along a second helical path about the central longitudinal axis. The first helical segment may include a first plurality of contact surfaces and the second helical segment may include a second plurality of contact surfaces such that when a torque is applied to the shaft, the first plurality of contact surfaces or the second plurality of contact surfaces bears against the other of the first plurality of contact surfaces or the second plurality of contact surfaces.

Further in the method according to the fifth aspect of the disclosure, additively manufacturing the flexible instrument may include depositing a first layer of powder onto a substrate; selectively heating the first layer of powder with a high energy beam to form first portions of the first end portion, the second end portion and the shaft; depositing a set of successive layers of the powder onto the first scanned layer; and selectively heating at least a portion of each of the layers of the first set of successive layers of the powder with the high energy beam to form additional portions of the first end portion, the second end portion and the shaft. The flexible instrument may be monolithic.

According to a sixth aspect of the disclosure, a method of manufacturing a flexible instrument may include forming a first portion of the surgical implant by: depositing a first quantity of material; fusing the first quantity of material to form a first fused layer; depositing a second quantity of material over the first fused layer; and fusing the second quantity of material to form a second fused layer; and then forming a second portion of the surgical implant by: depositing a third quantity of material; and fusing the third quantity of material to form a third fused layer; wherein upon formation of each portion, a flexible instrument is formed in its entirety, including a bendable shaft with a plurality of helical segments adapted to transfer torque.

Further in the method according to the sixth aspect of the disclosure, the flexible instrument may be formed in a single additive manufacturing process and may be fully assembled upon completion of the formation of fused layers.

According to a seventh aspect of the disclosure, a method of manufacturing a flexible instrument may include alternatingly depositing and heating successive layers of a first source material to form the flexible instrument, wherein the flexible instrument includes a first end portion, a second end portion opposite the first end portion, and a shaft. The shaft may extend from the first end portion to the second end portion along a central longitudinal axis. The shaft may include a first helical segment and a second helical segment. The first helical segment may extend from the first end portion to the second portion along a first helical path about the central longitudinal axis. The second helical segment may extend from the first end portion to the second end portion along a second helical path about the central longitudinal axis. The first helical segment may include a first plurality of contact surfaces and the second helical segment may include a second plurality of contact surfaces such that when a torque is applied to the shaft, the first plurality of contact surfaces or the second plurality of contact surfaces bears against the other of the first plurality of contact surfaces or the second plurality of contact surfaces.

### BRIEF DESCRIPTION OF THE DRAWINGS

FIG. 1 is a perspective view of an additively manufactured medical instrument according to an embodiment of the disclosure.

FIG. 2 is a bottom view of the medical instrument of FIG. 1.

FIG. 3 is a perspective bottom view of the medical instrument of FIG. 1.

FIG. 4 is a front view of a shaft of the medical instrument of FIG. 1 with a helical segment of the shaft highlighted.

FIG. 5 is a view of an isolated helical segment of the shaft of FIG. 4.

FIG. 6 is a close-up view of cutaway portion B of the helical segment of FIG. 5.

FIG. 7 is a front view of a shaft of the medical instrument of FIG. 1 with protrusions on one helical segment of the shaft highlighted.

FIG. 8 is a close-up partial view of the shaft of FIG. 7.

FIG. 9 is a close-up partial view of a portion of the medical instrument of FIG. 1.

FIG. 10 is a schematic diagram of the instrument of FIG. 1 including a handle and a cutting tip.

FIG. 11 is a front view of a shaft of a medical instrument according to another embodiment of the disclosure.

FIG. 12 is a close-up partial view of the shaft of FIG. 11.

### DETAILED DESCRIPTION

As used herein, the terms “about,” “generally,” “approximately,” and “substantially” are intended to mean that slight deviations from absolute are included within the scope of the term so modified. However, unless otherwise indicated, the lack of any such terms should not be understood to mean that such slight deviations from absolute are not included within the scope of the term so modified.

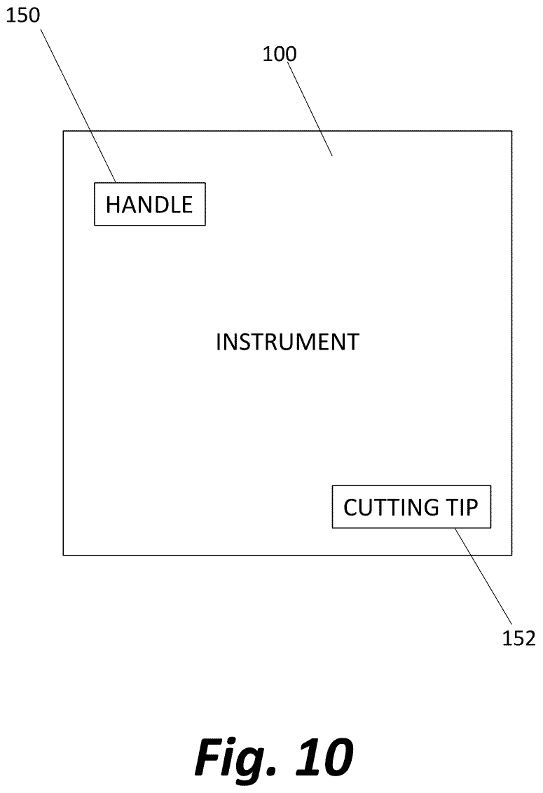

The present disclosure describes a structure of an instrument and a manufacturing process for creating the instrument, using a medical tool as an example and in particular a reamer, which is formed using a method of additive manufacturing (AM). For example, the described instrument may be used in arthroscopic applications such as reaming bones at a knee joint at any angle, such as an oblique angle. For example, the instrument may be coupled to or include a handle 150 or motorized device at a first end and may be coupled to or include a cutting tip 152 on a second end opposite the first end, as shown in FIG. 10. The instrument may be wielded by a user at the first end (e.g., handle 150) to navigate the second end having the cutting tip 152 to a desired target site, such as the surface of a bone, and the instrument 100 may be operated, e.g., power may be supplied to drive the shaft 110 so that the shaft 110 rotates to form a bore in the bone. In some scenarios, the target site may not be immediately accessible to the user wielding the instrument, and the second end of the instrument including the cutting tip 152 may need to be deformed and/or bent around other bones or portions of the patient's body to reach the target site. It should be understood that the disclosed process is not limited to the fabrication of medical tools and may be used for other types of additively manufactured objects. Similarly, while the described embodiments refer to an instrument, such reference should be understood as not limiting.

In some arrangements, one or more objects are fabricated by additive manufacturing means, such as by an SLS, SLM, or EBM process. The materials used to form the one or more objects may be, but are not limited to being, metal powder. Such metal powder may be, in some arrangements, any one or any combination of titanium, titanium alloys such as but not limited to Ti-6Al-4V, stainless steel, cobalt chrome alloys, silver, tantalum and niobium.

In some embodiments, the one or more objects include a base formed in a plurality of layers. A first layer of the instrument is formed on a substrate by depositing and then selectively scanning with a high energy beam, e.g., a laser or electron beam, a first layer of powder to sinter or melt and thereby fuse selective portions of the first layer of powder together. Successive layers of the powder are then deposited and selectively scanned with the high energy beam, layer by layer, to sinter or melt and thereby fuse selective portions of each of the successive layers of powder together over the first layer to form the instrument. During this process, each layer of the instrument being formed is supported by one or both of the substrate and the previously scanned layers as the powder is heated to be fused together, and the formed portions of the base have continued support while cooling.

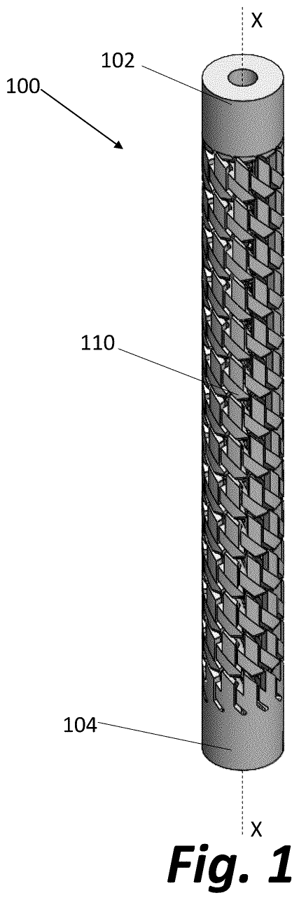

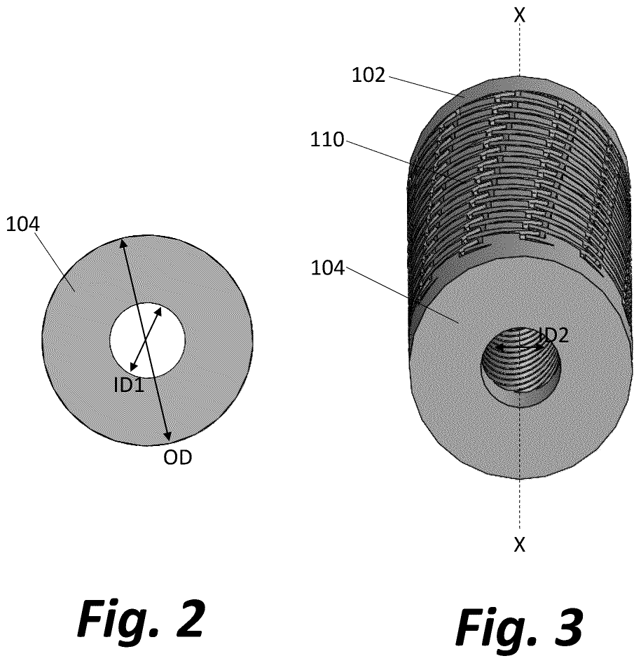

For example, in one embodiment shown in FIG. 1 (or FIG. 10 including handle 150 and cutting tip 152), the additively manufactured object is instrument 100 having first end portion 102 at a first extent of instrument 100, second end portion 104 at a second extent of instrument 100 opposite the first extent, and shaft 110 extending from first end portion 102 to second end portion 104. In FIG. 1, instrument 100 is generally cylindrical and extends in opposing directions along central longitudinal axis X defining a length spanning far ends of the first and second extents. In the depicted embodiment, instrument 100 is cannulated through its entire length along central longitudinal axis X, and includes first end portion 102, second end portion 104 and shaft 110. End portions 102, 104 of instrument 100 have an inner diameter ID₁ and an outer diameter OD as shown in FIG. 2, while instrument 100 has an outer diameter OD along shaft 110 and an inner diameter ID₂ in shaft 110. Inner diameter ID₂ is based on a thickness of helical segments 120, which are described in greater detail below. In the illustrated embodiment, ID₁ and ID₂ are approximately equal such that instrument 100 has a uniform inner diameter and a uniform outer diameter along its entire length. First and second end portions 102, 104 are generally solid and enclosed such that particles passable through shaft 110 cannot pass through outer surfaces of the end portions as shown in FIGS. 1-3.

Shaft 110 includes a plurality of helical segments 120 extending from first end portion 102 to second end portion 104. Each helical segment 120 revolves around central longitudinal axis X as each helical segment 120 extends along the length of shaft 110 from first end portion 102 to second end portion 104. Helical segments 120 extend parallel to one another, and each helical segment 120 is offset from the other helical segments 120 around the circumference of shaft 110 such that each distinct helical segment 110 occupies a unique physical space around the circumference of the shaft separate from the other helical segments while being disposed at approximately the same distance from central longitudinal axis X along an entire length of the helical segment.

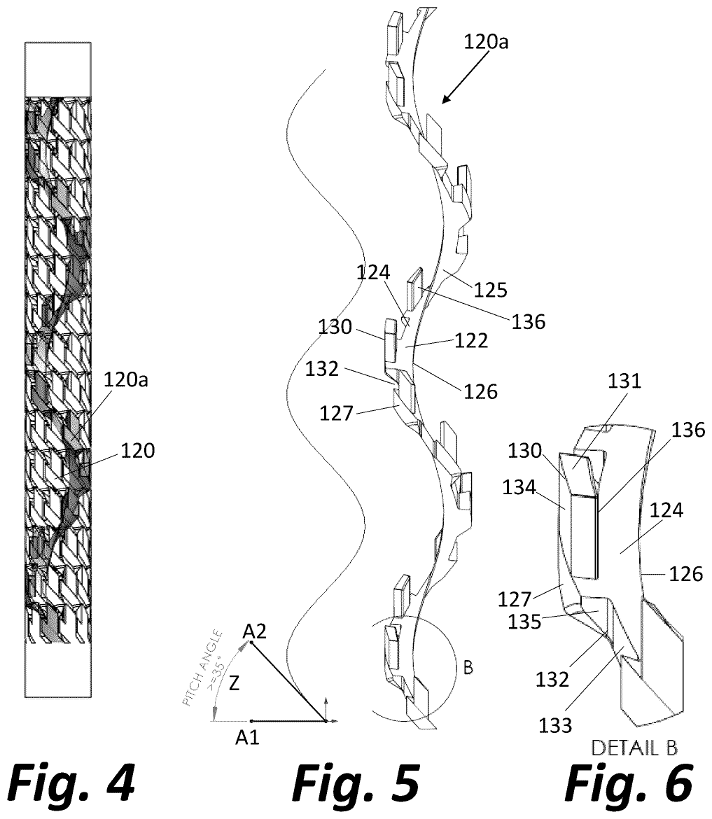

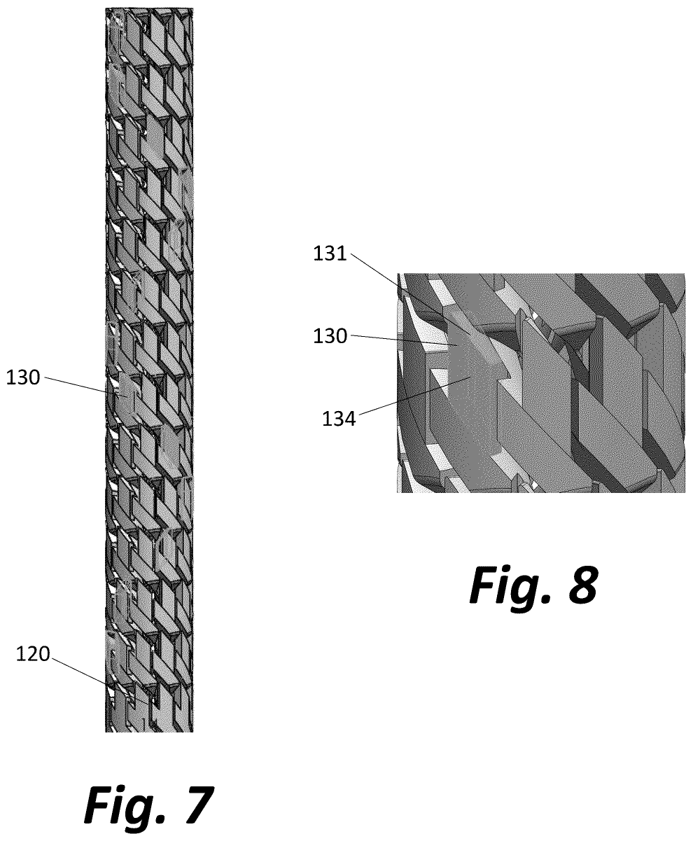

One helical segment 120a of instrument 100 is shown distinguished from the other helical segments within shaft 110 in FIG. 4 and helical segment 120a is illustrated by itself separated from the remainder of shaft 110 (e.g., other helical segments) in FIG. 5 for case of illustration. Because each helical segment 120 in shaft 110 is identical, helical segment 120a is representative of each of the other helical segments 120. In this manner, while the elements of helical segment 120a will be described in detail, such description is equally applicable to the other helical segments. Helical segment 120a includes base segment 122 extending along the length of helical segment 120 and includes an upper face 124 and a lower face 125 opposite upper face 124. Base segment 122 also includes radial inner face 126 and radial outer face 127 opposite radial inner face 126, each of the inner and outer faces separating the upper and lower faces. Helical segment 120a includes a plurality of teeth or protrusions 130 spaced at approximately equal distances apart from one another, each protrusion 130 extending from upper face 124 of base segment 122 in a direction generally toward first end portion 102 such that the protrusions may extend generally parallel to central longitudinal axis X and/or parallel to one another. As shown in FIGS. 6 and 8, protrusions 130 are cuboid in shape with an outward surface 134 facing away from axis X and an inward surface 136 opposite outward surface 134. Outward and inward surfaces of each protrusion 130 generally define a parallelogram. Remote from base segment 122 is end face 131 separating the outward and inward surfaces. End face 131 extends generally parallel to upper face 124 and lower face 125 of base segment 122. Helical segment 120a further defines a plurality of recesses 132 in radial outer face 127 of base segment 122 spaced at approximately equal distances apart from one another. Recesses 132 are spaced such that protrusions 130 and recesses 132 alternate along the length of base segment 122. Recesses 132 are also generally rectangular in shape when viewed from the side and are sized to receive protrusions 130 therein. Specifically, recesses 132 include a bottom surface 133 extending between wall surfaces 135, where the wall surfaces separate the bottom surface from radial outer face 127.

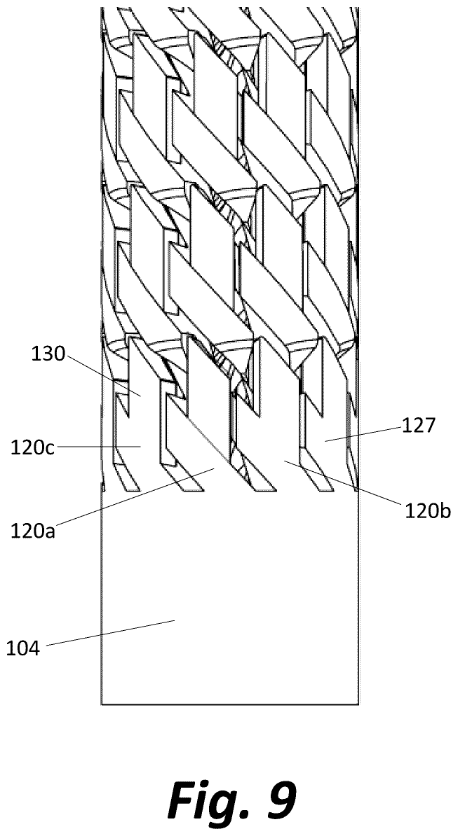

Referring back to FIG. 4, each protrusion 130 along a single helical segment 120 is sized, shaped and positioned to engage, mate and nest within a recess 132 of an adjacent helical segment. More specifically, the plurality of helical segments 120 are arranged such that each helical segment is adjacent to two other helical segments along the circumference of shaft 110, i.e., there is an adjacent helical segment on each side of every helical segment. For example, helical segment 120a (herein also referred to as “first helical segment 120a”) is adjacent to a second helical segment 120b and a third helical segment 120c as shown in FIG. 9, wherein second helical segment 120b is on a right side of first helical segment 120a (e.g., clockwise relative to first helical segment 120a when viewed from a bottom end of shaft 110) and third helical segment 120c is on a left side of first helical segment 120c (e.g., counter-clockwise relative to first helical segment 120a when view from a bottom end of shaft 110).

Each protrusion 130 along a single first helical segment (e.g., first helical segment 120a) is then nested within each recess 132 along a separate single helical segment positioned to the right or clockwise (e.g., second helical segment 120b) relative to the first helical segment. That is, all of protrusions 130 included on first helical segment 120a are nested within recesses 132 defined by second helical segment 120b. Similarly, all of protrusions 130 included on third helical segment 120c are nested within recesses 132 defined by first helical segment 120a. This pattern then continues around the circumference of shaft 110 to form a shaft made of interlocking helical segments. More specifically, for each protrusion 130 nested in a recess 132, inward surface 136 of protrusion 130 is proximate to or abuts bottom surface 133 of recess 132, while sidewalls of protrusion 130 are proximate to or abut wall surfaces 135 of recess 132, as shown in FIG. 8. In the depicted embodiment, the sidewalls of protrusion 130 extend vertically and parallel to each other as well as wall surfaces 135 of recess 132. A tolerance may be built into this interaction between parts so that some side-to-side movement between the protrusion and the recess may occur, in addition to the movement between the protrusion and the recess in a lengthwise direction, e.g., parallel to axis X, while the shaft is subject to various forces. In a preferred embodiment, bottom surface 133 of recess 132 may span a distance along a length direction of the helical segment that is between about 0.15 and 0.5 millimeters greater than a corresponding size of protrusion 130. In other words, the distance between wall surfaces 135 of recess 130 may be between about 0.15 and 0.5 millimeters greater than the distance between the sidewalls of protrusion 130. Thus, when the protrusion is seated in the recess, the difference in lengthwise dimension between the protrusion and the recess provides the above-described tolerance and facilitates side-to-side movement of the protrusion with respect to the recess. The geometric relationships between surfaces of protrusion 130 and recess 132 as well as the above-described tolerance preserve the ability of instrument 100 to transfer torque, particularly when bent or flexed at oblique angles. Additionally, it is contemplated that if the tolerance is large enough (e.g., if the distance between wall surface 135 is sufficiently greater than the distance between the sidewalls of protrusion 130), it may be possible for first end portion 102 to rotate slightly about axis X relative to second end portion 104. The greater the tolerance that exists between protrusions 130 and recesses 132, the more relative rotation between the end portions will be possible, and vice versa.

In some examples, and as shown in FIG. 9, radial outer face 127 of helical segments 120 is flush with an outer surface of second end portion 104 (which is also true of the outer surface of first end portion 102). That is, there is no gap, lip, shoulder, connection point, etc. formed between radial outer surface 127 of helical segments 120 and the outer surfaces of first and second end portions 102, 104. This smooth transition between shaft 110 and end portions 102, 104 may result from fabricating the instrument monolithically using additive manufacturing techniques.

It is contemplated that in alternative variations of instrument 100, the helical segments may be shaped such that at least some of the helical segments have different shapes and/or structures. Some helical segments may have a different structure than others, or all helical segments may be distinct from one another. Protrusions 130 and recesses 132 need not be spaced equal distances apart nor exactly alternating. For example, protrusions 130 may be intermittently positioned along helical segment 120, with recesses 132 correspondingly positioned intermittently along an adjacent helical segment 120 to mate with the protrusions. In some examples, protrusions 130 may be grouped together and recesses 132 may be grouped together such that each helical segment includes two, three, or any number of consecutive protrusions, which are then separated by one recess or a plurality of consecutive recesses (e.g., a pattern of two consecutive protrusions alternating with two consecutive recesses), so long as each protrusion corresponds to a recess on an adjacent helical segment. In other examples, the pattern of protrusions and recesses along the helical segment may be completely randomized, again, so long as each protrusion corresponds to a recess on an adjacent helical segment.

Protrusions may extend from lower face 125 of base segment 122 rather than upper face 124, and recesses may be defined along radial inner face 126 of base segment 122 rather than radial outer face 127. In other examples, the respective protrusions and recesses may be oriented at an angle relative to a longitudinal axis on an outer surface of shaft 110. Shaft 110 may include as few as two helical segments 120, or any number greater than two. In the depicted embodiment, shaft 110 includes 11 helical segments. In one specific example where an instrument has an outer diameter OD smaller than that of instrument 100 in the illustrated embodiment, the instrument may include 9 helical segments to accommodate the size and desired flexibility. Other quantities of helical segments are also contemplated to further modify the size and flexibility of the instrument. Protrusions 130 may be formed in any shape and may even be varied to modify the ability of shaft 110 to flex. For example, protrusions 130 may be shaped with faces that are triangular, rounded or extend from base segment 122 at an oblique angle or include a curvature as they extend from base segment 122. Any design parameters of instrument 100 may be adjusted to allow for larger flexing angles, increased torque capacity, modified diameters, etc.

First end portion 102 and second end portion 104 may be porous or perforated, or may be generally non-porous but occupying a portion of an overall volume of the instrument much smaller or larger than what is shown in the figures. In some examples, inner diameter and thickness may vary along the length of the instrument. For instance, shaft 110 may have an inner diameter ID₂ larger than the inner diameter ID₁ of end portions 102, 104, while maintaining the same outer diameter OD, thus causing the thickness of the end portions to be greater than the thickness of the shaft. In some embodiments, first end portion 102 and second end portion 104 may be solid all the way through and/or may not be cannulated as shown in the figures. Instrument 100 may be formed of titanium, 3164 steel, or other materials having a similar modulus of elasticity, which promotes the flexibility of shaft 110. In other embodiments, instrument 100 may be formed of plastic or other polymers, which may be advantageous for lower-torque applications, decreasing the weight or cost of the instrument and/or employing as single-use or disposable instruments.

It is contemplated that outer diameter OD may measure between about 10 millimeters and about 15 millimeters, and inner diameters ID₁ and ID₂ of end portions 102, 104 may measure between about 3 millimeters and about 6 millimeters. In a preferred embodiment, outer diameter OD measures about 12 millimeters and inner diameters ID₁ and ID₂ measure about 4.3 millimeters, but the design may be altered to allow for smaller or larger diameters, e.g., to modify the size of the cannulation. For instance, in a smaller embodiment described above having nine helical segments, the outer diameter may measure approximately 6 millimeters and the inner diameters may measure 3 millimeters. As shown in FIG. 5, a pitch angle Z is the angle defined between a line A1 extending along the plane perpendicular to central longitudinal axis X and line A2 extending tangential to helical segment 120a and intersecting with line A1, and is indicative of the pitch of helical segments, e.g., a distance along the length or longitudinal distance covered by the helical segment upon one full revolution of the helical segment around central longitudinal axis X. In some examples, helical segments 120 may have a pitch angle Z between about 30 degrees and about 50 degrees, although any pitch angle is contemplated. In some preferred embodiments, helical segments 120 are formed with a pitch angle Z of at least 35 degrees relative to a plane extending perpendicular to central longitudinal axis X. In examples where pitch angle Z is greater than 35 degrees, shaft 110 will have greater performance in terms of torque transfer than lower pitch angles, whereas in examples where pitch angle Z is less than 35 degrees, shaft 110 will have greater flexibility than higher pitch angles.

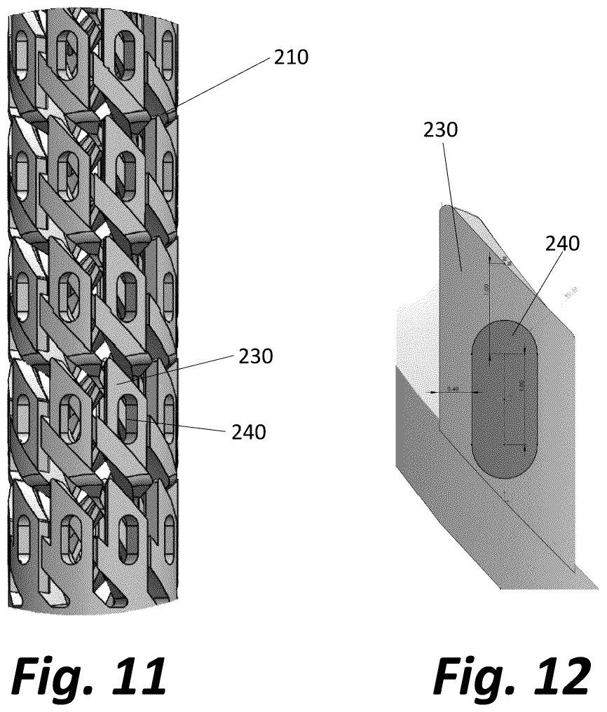

In some examples, such as the alternate embodiment shown in FIGS. 11-12, wherein like reference numerals refer to like parts but in the −200 series of numbering, instrument 100 may include a shaft 210 having protrusions 230 which define an aperture 240 extending therethrough. That is, each protrusion 230 may define a single aperture 240 as shown. Apertures 240 are generally stadium-shaped and extend vertically when shaft 210 is oriented upright as in FIG. 11. In the illustrated embodiment, aperture 240 has an inner rectangular portion defining a length of approximately 1 millimeter, and the aperture further includes semi-circular portions on opposing sides of the inner rectangular portion each having a radius of curvature measuring approximately 0.38 millimeters. Further, aperture 240 is spaced approximately 0.4 millimeters from an edge of protrusion 230 in the width direction (i.e., orthogonal to the length direction). It is contemplated that shaft 210 may be utilized in various operations or with different patients and may require cleaning between each use for sanitation purposes. While undergoing such cleaning and autoclave cycles, apertures 240 promote case of cleaning by allowing materials caught in the shaft, e.g., between protrusions, to be dislocated and removed from the shaft.

Alternatively, each protrusion 230 may define a plurality of apertures, or some protrusions may define one or several apertures whereas other protrusions on shaft 210 are solid and do not have any apertures extending therethrough. The apertures may define any shape and size, such as a circle, rectangle, etc., and may be positioned any distance from the edges of the protrusions.

In some examples, instrument 100 may be packaged or provided in a kit with any combination of other tools and instruments with which instrument 100 may be used. For instance, a kit may include instrument 100 along with a handheld or motorized device to which instrument 100 may be coupled, a sharpened or cutting tip 152 or a plurality of cutting tips in varying sizes which may be coupled to instrument 100, or any combination of the above-noted components.

In another aspect, the present disclosure relates to a method of manufacturing an object, such as a surgical instrument. Instrument 100 is formed layer-by-layer using an additive layer manufacturing (AM), i.e., 3D printing, process so no separate connection mechanism is necessary to bring together any of the elements of the instrument, such as the helical segments of the shaft or the shaft with the first and second end portions. Through this process, instrument 100 may be formed in a single stage process. Further, the instrument that is formed may be monolithic in that end portions 102, 104 and shaft 110 are all part of an integrally formed structure. In some examples, AM processes are powder-bed based and involve one or more of selective laser sintering (SLS), selective laser melting (SLM), and electron beam melting (EBM), as disclosed in U.S. Pat. Nos. 7,537,664; 8,728,387; 9,180,010; and 9,456,901, the disclosures of which are hereby incorporated by reference in their entireties herein.

The AM process used to form instrument 100 produces a shaft 110 having overlapping and interlocking helical segments 120 with protrusions 130 of one helical segment nested in and radially layered in a recess 132 on a separate helical segment. The interaction between the respective protrusions on one helical segment with the surfaces of the recess in an adjacent helical segment, along with similar interactions for all helical segments on the shaft, allows torque to be effectively transmitted by the instrument when the shaft is flexed or bent with respect to the central longitudinal axis.

It is noted that instrument 100 is described herein as being additively manufactured in the orientation as shown in FIG. 1, however the instrument is not limited to such an orientation. As shown, instrument 100 is oriented vertically or upright, with first end portion 102 comprising an upper portion, shaft 110 comprising a middle portion, and second end portion 104 comprising a lower end portion of the instrument. In other examples, instrument 100 may be manufactured on its side such that the length of the instrument extends generally horizontally rather than vertically. In one embodiment of forming instrument 100, a first layer of metal powder may be deposited on a build plate of the manufacturing machine and that first layer of powder is selectively scanned by an electron beam, in this example, to form second end portion 104 nearest the built plate, which supports subsequent build layers as they are formed directly on top of the initial layer. It should be noted that less material is needed while manufacturing shaft 110 of instrument 100 as compared to an equal-length portion of either first end portion 102 or second end portion 104 due to the porosity and perforations included in shaft 110 as described above. Furthermore, in examples where shaft 110 has an inner diameter greater than an inner diameter of first and second portions 102, 104, the shaft again requires even less material to be manufactured as compared to equal-length portions of the end portions due to its reduced thickness.

In some embodiments, the method of additively manufacturing a three-dimensional object may be performed in a cycle. A cycle may begin with depositing a first layer of powder onto a substrate, which may be a start plate. The first layer of powder may be selectively scanned with a high energy beam such as a laser or electron beam to sinter or melt the first layer of powder and to form initial portions of both the base of the object, e.g., second end portion 104. After at least a first layer is scanned, successive layers of powder may be deposited and each such successive layer may be selectively scanned in a manner substantially similar to the first layer. The machine depositing the powder may be programmed to deposit the powder in locations corresponding to the shape of the three-dimensional object programmed into the machine (e.g., instrument 100). Additional layers may be deposited and scanned to form shaft 110 and first end portion 102 until instrument 100 is fully formed, completing one full AM cycle.

In another aspect, the instrumentation described in the present disclosure, such as instrument 100, may be used, for example, to repair bone or as part of an operation to repair soft tissue. Specific repair procedures may include, among others, procedures to repair ligaments and tendons in a knee, hip, ankle, foot, shoulder, elbow, wrist, hand, spine or any other area of human anatomy.

In one embodiment, instrument 100 may be used in arthroscopic repairs of an ACL in a knee joint. For instance, after soft tissue is distracted to expose an area to be operated upon, instrument 100 may be used to ream a tibial and/or femoral bone tunnel. The bone tunnel may then be used, for example, to pass an ACL replacement graft through the bone tunnel as part of a repair. Examples of procedures that may be performed using instrument 100 are described in U.S. Pat. No. 9,795,398, the disclosure of which is hereby incorporated by reference herein in its entirety. During repair procedures such as those described above, obstructions within the body or constraints on an extent of the surgical access may interfere with the surgeon's direct line of sight to the bone to be reamed for the formation of a tunnel. This may pose a difficulty because such a circumstance would require the instrument to be inserted from an angle or direction other than linear and head on. Such need for angulation of the instrument is provided through the flexible structure of instrument 100, where instrument 100 is configured to bend based on the operative communication of the helical segments and the relative dimensions of the interacting components, i.e., protrusions and recesses, as noted above. In some examples, instrument 100 may be manually bent by the surgeon using a hand or a secondary tool positioned to grip or otherwise engage the instrument. In other examples, a guidewire may initially be placed into the bone tunnel, and then instrument 100 may be inserted over the guidewire along a linear alignment to ream a small portion of the bone. Once the bone is reamed sufficiently, a distal end of the instrument is anchored into the bone, which allows the surgeon to manipulate a proximal end of the instrument to bend or flex the instrument as necessary to relieve tension in the distracted area while also continuing to apply torque at the proximal end so that the reaming may continue to fully drill the bone tunnel. These exemplary methods of using the instrumentation may be performed when the knee is positioned at a “normal” flexion, for example, at ninety degrees. These methods may also reduce the need for a surgeon to hyperflex the knee, thereby facilitating repairs knees that cannot undergo hyperflexion.

As described above, instrument 100 is additively manufactured as a monolithic instrument, and therefore offers a single component design feature that is a self-supporting structure that allows the ability to transfer torque at a flexed angle without the need for any additional manufacturing steps to be applied to allow the shaft to flex or transfer torque. The nested profiled of the protrusions within the recesses as created by additive manufacturing allows formation of a different geometry than conventional tools formed by subtractive manufacturing such as laser cutting. The interlocking of protrusions with recesses between helical segments allows torque to be transmitted along the length of the instrument even when the instrument is flexed or bent. The engaging faces of the protrusions and corresponding recesses are generally parallel to one another when the instrument is at rest, which helps with the transfer of torque through the instrument. Furthermore, the dimensions of the protrusions with respect to the corresponding recesses allow for sufficient movement and sliding of the protrusion within the recess so that the overall structure can flex about the central longitudinal axis. The material and structure of instrument 100 contribute to the instrument being biased to its resting configuration as shown in FIG. 1, such that instrument 100 behaves elastically and bending or flexing of shaft 110 causes spring-like behavior to bring the shaft back to its resting configuration. The porosity or perforation of shaft 110 allows for easy and thorough cleaning of the instrument.

It is to be further understood that the disclosure set forth herein includes any possible combinations of the particular features set forth above, whether specifically disclosed herein or not. For example, where a particular feature is disclosed in the context of a particular aspect, arrangement, configuration, or embodiment, that feature can also be used, to the extent possible, in combination with and/or in the context of other particular aspects, arrangements, configurations, and embodiments of the disclosure.

Although particular embodiments have been described herein, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A flexible instrument comprising:
a first end portion;
a second end portion opposite the first end portion; and
a shaft extending from the first end portion to the second portion along a central longitudinal axis, the shaft including:
a first helical segment extending from the first end portion to the second end portion along a first helical path about the central longitudinal axis; and
a second helical segment extending from the first end portion to the second end portion along a second helical path about the central longitudinal axis,
wherein the first helical segment includes a first plurality of contact surfaces and the second helical segment includes a second plurality of contact surfaces such that when a torque is applied to the shaft, the first plurality of contact surfaces or the second plurality of contact surfaces bears against the other of the first plurality of contact surfaces or the second plurality of contact surfaces.

2. The flexible instrument of claim 1, wherein the first helical segment includes a protrusion extending from the first helical segment.

3. The flexible instrument of claim 2, wherein the second helical segment defines a recess on a face of the second helical segment configured to receive the protrusion of the first helical segment.

4. The flexible instrument of claim 1, wherein the first plurality of contact surfaces further comprises a plurality of protrusions extending from the first helical segment, wherein the plurality of protrusions is spaced apart from each other along a length of the first helical segment.

5. The flexible instrument of claim 4, wherein the plurality of protrusions extends from the first helical segment in parallel directions.

6. The flexible instrument of claim 4, wherein the second plurality of contact surfaces further comprise a plurality of recesses on a face of the second helical segment, the plurality of recesses spaced apart from each other along a length of the second helical segment.

7. The flexible instrument of claim 6, wherein each one of the plurality of protrusions is positioned within a corresponding one of the plurality of recesses.

8. The flexible instrument of claim 1, wherein the shaft is perforated defining gaps between the first helical segment and the second helical segment.

9. The flexible instrument of claim 1, wherein the first end portion, the shaft, and the second end portion are monolithic.

10. The flexible instrument of claim 3, wherein the recess is sized and shaped to allow movement of the protrusion relative to the recess when the instrument is flexed.

11. A flexible instrument comprising:
a first end portion;
a second end portion opposite the first end portion; and
a cannulated shaft extending from the first end portion to the second end portion such that the cannulated shaft, the first end portion and the second end portion are aligned along a central longitudinal axis,
wherein the cannulated shaft has a neutral configuration where the central longitudinal axis is linear and the cannulated shaft is deformable into a bent configuration such that the central longitudinal axis is curved, and
wherein the cannulated shaft is adapted to transfer torque between surfaces of interfacing subportions of the cannulated shaft when the cannulated shaft is rotated about the central longitudinal axis, the surfaces of the interfacing subportions being proximate an outer surface of the cannulated shaft and remote from the central longitudinal axis.

12. The flexible instrument of claim 11, wherein the surfaces of the interfacing subportions being on respective planes that are parallel to the central longitudinal axis.

13. The flexible instrument of claim 11, wherein the cannulated shaft is formed of a plurality of segments extending from the first end portion to the second end portion.

14. The flexible instrument of claim 11, wherein each of the plurality of segments revolve around the central longitudinal axis as each segment extends from the first end portion to the second portion.

15. The flexible instrument of claim 11, wherein adjacent ones of the plurality of segments are interconnected.

16. The flexible instrument of claim 15, wherein the interconnection is sliding contact between a first of the plurality of segments and a second of the plurality of segments.

17. The flexible instrument of claim 11, wherein each of the plurality of segments includes a protrusion along a length and defines a recess separated from the protrusion along the length.

18. The flexible instrument of claim 17, wherein the protrusion of each of the plurality of segments mates with the recess of an adjacent one of the plurality of segments.

19. The flexible instrument of claim 11, further comprising:
a handle at the first end portion or the second end portion; and
a cutting tip at the other of the first end portion or the second end portion,
wherein the cutting tip is configured to resect bone.

20. A flexible instrument comprising:
A first end portion;
A second end portion opposite the first end portion; and
A shaft extending along a central longitudinal axis from the first end portion to the second end portion, the shaft including a plurality of parallel segments extending from the first end portion to the second end portion, wherein each of the parallel segments is shaped to revolve around the central longitudinal axis as the segments extend from the first end portion the second portion.
